(19) United States
(12) Patent Application Publication
(10) Pub. No.: US 2018/0257952 A1
(43) Pub. Date: Sep. 13, 2018

(54) FLUID STERILIZATION APPARATUS

(71) Applicant: Nikkiso Co., Ltd, Tokyo (JP)

(72) Inventors: Ayako WADA, Hakusan-Shi Ishikawa (JP); Hideki ASANO, Hakusan-Shi Ishikawa (JP)

(21) Appl. No.: 15/913,100

(22) Filed: Mar. 6, 2018

(30) Foreign Application Priority Data
Mar. 9, 2017 (JP) 2017-045275

(51) Int. Cl. C02F 1/32; A61L 2/00; A61L 2/26; A61L 9/20

(52) U.S. Cl. CPC C02F 1/32; A61L 2/0047; A61L 2/26; A61L 9/20; C02F 2201/3222; A61L 2202/21; A61L 2209/12; C02F 2201/3228; C02F 2303/04; A61L 2202/11

(57) ABSTRACT

A fluid sterilization apparatus is provided with: a light source that has a semiconductor light-emitting element that emits ultraviolet light; and a flow passage that is formed so as to allow a fluid subject to sterilization to pass through and allow the fluid to be irradiated with ultraviolet light. The flow passage is formed such that the flowing direction of the fluid changes in a plane that faces a light-emitting surface of the semiconductor light-emitting element.

### CROSS-REFERENCE TO RELATED APPLICATIONS

This application is based upon and claims the benefit of priority from the prior Japanese Patent Application No. 2017-045275, filed on Mar. 9, 2017, the entire contents of which are incorporated herein by reference.

### BACKGROUND OF THE INVENTION

### 1. Field of the Invention

The present invention relates to a fluid sterilization apparatus.

### 2. Description of the Related Art

In the related art, apparatuses have been suggested that sterilize a fluid flowing inside a flow passage by irradiation with ultraviolet light from around the flow passage. For example, an ultraviolet sterilization water purification apparatus is known where an ultraviolet LED unit that irradiates water with ultraviolet light is arranged in a container for storing water or on an internal wall of a flow passage for water flow (see JP 2011-16074).

Since an ultraviolet light irradiation range of a single ultraviolet LED unit is limited, the above-stated ultraviolet sterilization water purification apparatus is formed to be able to sterilize water more efficiently by arranging a plurality of ultraviolet LED units around a single flow passage. However, if the flow rate is increased in order to improve the processing efficiency, sufficient sterilization may not be able to be performed. Meanwhile, if the length of the flow passage is increased in order to perform sufficient sterilization such that the number of ultraviolet LED units arranged around the flow passage is increased, the size of the apparatus will end up being increased.

### SUMMARY OF THE INVENTION

In this background, one of exemplary purposes of the present invention is to provide a space-saving fluid sterilization apparatus.

A fluid sterilization apparatus according to one embodiment of the present invention is provided with: a light source that has a semiconductor light-emitting element that emits ultraviolet light; and a flow passage that is formed so as to allow a fluid subject to sterilization to pass through and allow the fluid to be irradiated with ultraviolet light. The flow passage is formed such that the flowing direction of the fluid changes in a plane that faces a light-emitting surface of the semiconductor light-emitting element.

### BRIEF DESCRIPTION OF THE DRAWINGS

Embodiments will now be described, by way of example only, with reference to the accompanying drawings that are meant to be exemplary, not limiting, and wherein like elements are numbered alike in several figures, in which:

FIG. 1 is a schematic diagram showing a schematic configuration of a fluid sterilization apparatus according to a first embodiment;

FIG. 2 is a cross-sectional view taken along A1-A1 of FIG. 1;

FIG. 3 is a horizontal cross-sectional view of a fluid sterilization apparatus according to a second embodiment;

FIG. 4 is a schematic diagram showing a schematic configuration of a fluid sterilization apparatus according to a third embodiment;

FIG. 5A is a cross-sectional view taken along A2-A2 of FIG. 4, and FIG. 5B is a cross-sectional view taken along A3-A3 of FIG. 4;

FIG. 6 is a diagram showing the layout of a light source of the fluid sterilization apparatus according to the third embodiment;

FIG. 7A is a schematic diagram showing a schematic configuration of a fluid sterilization apparatus according to an exemplary variation of the third embodiment, and FIG. 7B is a cross-sectional view taken along A4-A4 of FIG. 7A;

FIG. 8A is a top view of a first flow passage in a fluid sterilization apparatus according to a fourth embodiment, and FIG. 8B is a top view of a second flow passage in the fluid sterilization apparatus according to the fourth embodiment;

FIG. 9A is a schematic diagram showing a schematic configuration of a fluid sterilization apparatus according to a fifth embodiment, and FIG. 9B is a cross-sectional view taken along A5-A5 of FIG. 9A; and

FIG. 10A is a schematic diagram showing a schematic configuration of a fluid sterilization apparatus according to a sixth embodiment, and FIG. 10B is a cross-sectional view taken along A6-A6 of FIG. 10A.

### DETAILED DESCRIPTION OF THE INVENTION

A fluid sterilization apparatus according to one embodiment of the present invention is provided with: a light source that has a semiconductor light-emitting element that emits ultraviolet light; and a flow passage that is formed so as to allow a fluid subject to sterilization to pass through and allow the fluid to be irradiated with ultraviolet light. The flow passage is formed such that the flowing direction of the fluid changes in a plane that faces a light-emitting surface of the semiconductor light-emitting element.

According to this embodiment, by forming the flow passage in such a manner that the flowing direction of the fluid changes, the length of the flow passage to be arranged in a region irradiated with ultraviolet light can be made to be longer compared to a case where the flow passage is a straight pipe. In other words, since the flow passage can be arranged efficiently in the region irradiated with ultraviolet light, the size of the fluid sterilization apparatus can be reduced.

The flow passage may have a first flow passage on a first stage having an inlet port for the fluid and a second flow passage on a second stage having an outlet port for the fluid. An end portion on the side opposite to the inlet port of the first flow passage and an end portion on the side opposite to the outlet port of the second flow passage may be connected to each other. This allows the fluid passing through the second flow passage or the first flow passage to be irradiated with ultraviolet light that has passed through the first flow passage or the second flow passage.

The first flow passage may be arranged above the second flow passage so as to overlap with the second flow passage. This allows the fluid that has passed through the first flow passage to flow into the second flow passage due to the fluid's own weight.

The light source may have: a first light source for irradiation with ultraviolet light from one side of the flow passage; and a second light source for irradiation with ultraviolet light from the other side of the flow passage. This allows for irradiation with ultraviolet light from both sides of the flow passage. Thus, in comparison with irradiation with ultraviolet light from one side, the irradiation intensity of ultraviolet light for the fluid passing through a predetermined interval can be higher, and the irradiation time can be shortened. In other words, since a flow passage that is arranged in a region irradiated with ultraviolet light can be shortened or the flow rate can be increased (the inner diameter of the flow passage can be decreased), the size of the apparatus can be reduced.

The fluid sterilization apparatus may further comprise a housing that houses the light source and the flow passage. On the inside of the housing, a reflective portion that reflects ultraviolet light emitted from the semiconductor light-emitting element may be provided.

The flow passage may be formed such that at least a portion thereof has a spiral shape or a swirling shape. This allows for the arrangement of the flow passage with a high space usage efficiency.

The flow passage may be formed of a tube. This allows the flow passage to be realized using a simple structure compared to a case where the flow passage is formed using a combination of a plurality of parts.

The tube may be made of a fluorine-based resin or a silicone based resin. This allows the flow passage having a desired shape to be formed with relative ease using a light resistant material.

The flow passage may be formed of a continuous tube having a folded part in the middle, the folded part being arranged near the center of the fluid sterilization apparatus. Thereby, the single flow passage where both end portions of the flow passage are located close to each other can be formed easily.

The flow passage may be formed such that the inner diameter of the flow passage in the middle of the flow passage is larger than the inner diameter of the flow passage near an inlet port or an outlet port for the fluid. This allows the flow rate in the middle of a flow passage to be decreased. Thus, for example, by devising the arrangement of a light source so that the intensity of ultraviolet light irradiation in the middle of the flow passage is increased, the fluid can be efficiently sterilized.

In the light source, a plurality of semiconductor light-emitting elements may be arranged in a line or a matrix of m by n (m is an integer of one or more and n is an integer of two or more). This allows sufficient sterilization performance to be achieved all through the flow passage.

The light source may have a first semiconductor light-emitting element arranged so as to face the central part of the flow passage and a second semiconductor light-emitting element arranged near the inlet port or near the outlet port. The output of the second semiconductor light-emitting element is larger than the output of the first semiconductor light-emitting element. Thereby, unevenness in the sterilization performance can be prevented all through the flow passage.

Optional combinations of the aforementioned constituting elements, and implementations of the invention in the form of methods, apparatuses, and systems may also be practiced as additional modes of the present invention.

The invention will now be described by reference to the preferred embodiments. This does not intend to limit the scope of the present invention, but to exemplify the invention.

Described below is an explanation of the embodiments of the present invention with reference to figures. In the explanation of the figures, like numerals represent like constituting elements, and duplicative explanations will be omitted appropriately. The structure described below is by way of example only and does not limit the scope of the present invention.

### First Embodiment

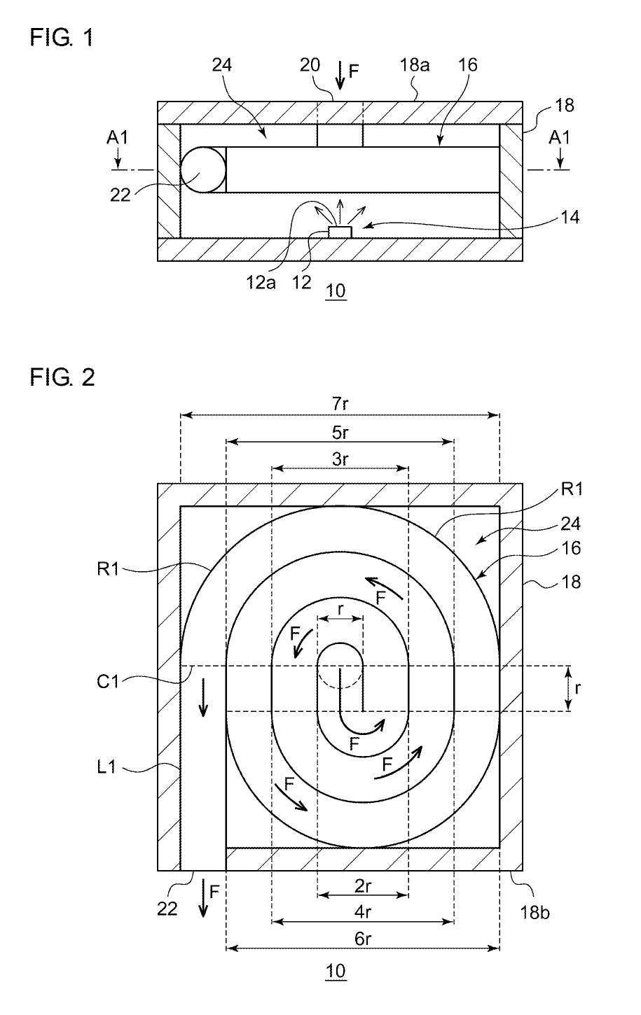

FIG. 1 is a schematic diagram showing a schematic configuration of a fluid sterilization apparatus according to a first embodiment. FIG. 2 is a cross-sectional view taken along A1-A1 of FIG. 1.

A fluid sterilization apparatus 10 is provided with: a light source 14, which has a semiconductor light-emitting element 12 that emits ultraviolet light; a flow passage 16, which is formed so as to allow a fluid F subject to sterilization to pass through and allow the fluid to be irradiated with ultraviolet light; and a housing 18, which houses the light source 14 and the flow passage 16. The fluid sterilization apparatus 10 may be provided with a pump serving as a driving means for delivering a fluid, a control valve for controlling the stopping and resuming of the delivery of the fluid, a control unit that controls the driving of a semiconductor light-emitting element and the pump and the opening and closing of the control valve, and the like. The fluid F subject to sterilization includes, for example, a liquid such as water and drugs and a gas such as the air and oxygen.

A semiconductor light-emitting element 12 according to the present embodiment is a light-emitting diode that emits ultraviolet light. The light-emitting diode according to the present embodiment has a substrate formed of sapphire, an AlGaN-based light-emitting layer, and a lattice mismatch buffer layer formed of AlN stacked between the substrate and the light-emitting layer. This allows for the achievement of a compact and highly-efficient sterilization apparatus. The semiconductor light-emitting element 12 preferably emits ultraviolet light whose peak light emission wavelength is in a range of 250 nm to 350 nm and more preferably emits ultraviolet light whose peak light emission wavelength is in a range of 260 nm to 300 nm. For the light-emitting diode according to the present embodiment, for example, a light-emitting diode whose peak light emission wavelength is 285 nm is used. This allows for an increase in sterilization effects.

In the center of an upper surface 18a of the housing 18, an inlet port 20 through which the fluid flows in is formed. On a side surface 18b of the housing 18, an outlet port 22 through which the fluid flows out is formed. The flow passage 16 is provided in the downward direction toward a sterilization chamber 24 inside the housing 18 from the inlet port 20, circles outwardly in a swirling shape (or a spiral shape) after changing the direction to the horizontal direction inside the sterilization chamber 24, and is eventually connected to the outlet port 22 formed at the side surface 18b of the housing 18.

The flow passage 16 is formed such that the flowing direction of the fluid F changes in a plane (for example, A1-A1 cross-sectional surface) that faces a light-emitting surface 12a of the semiconductor light-emitting element 12. The specific length L (the length from a position directly below the inlet port 20 to a connecting part C1 connecting a straight passage L1 immediately before the outlet port 22 and a curved passage R1) of the flow passage 16 is, as shown in FIG. 2, expressed as follows based on the outer circumference of the flow passage 16: 6r (six straight passages)+14πr (seven semicircles=πr/2+2πr/2+3πr/2+ . . . 7πr/2). In other words, in the sterilization chamber 24 whose one side is 7r, the flow passage 16 having a length of (6r+14πr) or more can be arranged. On the other hand, in a case where the flow passage is a straight pipe, only a flow passage having a length of 7r can be arranged in the sterilization chamber 24.

As described, the configuration of the flow passage 16 according to the present embodiment allows the length of a flow passage to be arranged in a region (sterilization chamber 24) irradiated with ultraviolet light to be longer compared to a case where the flow passage is a straight pipe. In other words, since a flow passage can be arranged efficiently in the region irradiated with ultraviolet light, the size of the fluid sterilization apparatus 10 can be reduced. The expression “formed such that the flowing direction of the fluid changes” includes, for example, a case where a continuous curve is provided or a partial curve (e.g., the curved passage R1 shown in FIG. 2) is provided in a portion of the flow passage, a case where a portion of the flow passage is curved in a discontinuous manner, a case where the flow passage is bent in a crank shape (e.g., at a right angle), a case of a bellows-like shape where folded parts exist repeatedly, and the like.

Also, as in the case of the fluid sterilization apparatus 10 according to the present embodiment, forming the flow passage 16 in such a manner that at least a portion thereof has a spiral shape or a swirling shape allows for the arrangement of the flow passage with a high space usage efficiency. As a result, a small and space-saving fluid sterilization apparatus 10 can be provided. Further, since the swirling-shape arrangement is similar to the spreading of ultraviolet light emitted from the semiconductor light-emitting element 12, the entire flow passage can be irradiated with ultraviolet light only by arranging one or a small number of semiconductor light-emitting elements 12 in the center of the housing 18.

The flow passage 16 according to the present embodiment is formed of a tube. This allows the flow passage to be realized using a simple structure compared to a case where the flow passage is formed using a combination of a plurality of parts. Regarding the shape of a tube, for example, there can be a case where the inner circumference and the outer circumference are circular or elliptical and a case where at least one of the inner circumference and the outer circumference is polygonal.

The tube that forms the flow passage 16 is preferably made of a resin that is transparent to ultraviolet light such as a fluorine-based resin or a silicone based resin. The fluorine-based resin includes, for example, perfluoroalkoxy alkane (PFA) and polytetrafluoroethylene (PTFE), which have an amorphous crystalline structure. This allows a flow passage having a desired shape to be formed with relative ease using a light resistant (ultraviolet light resistant) material.

### Second Embodiment

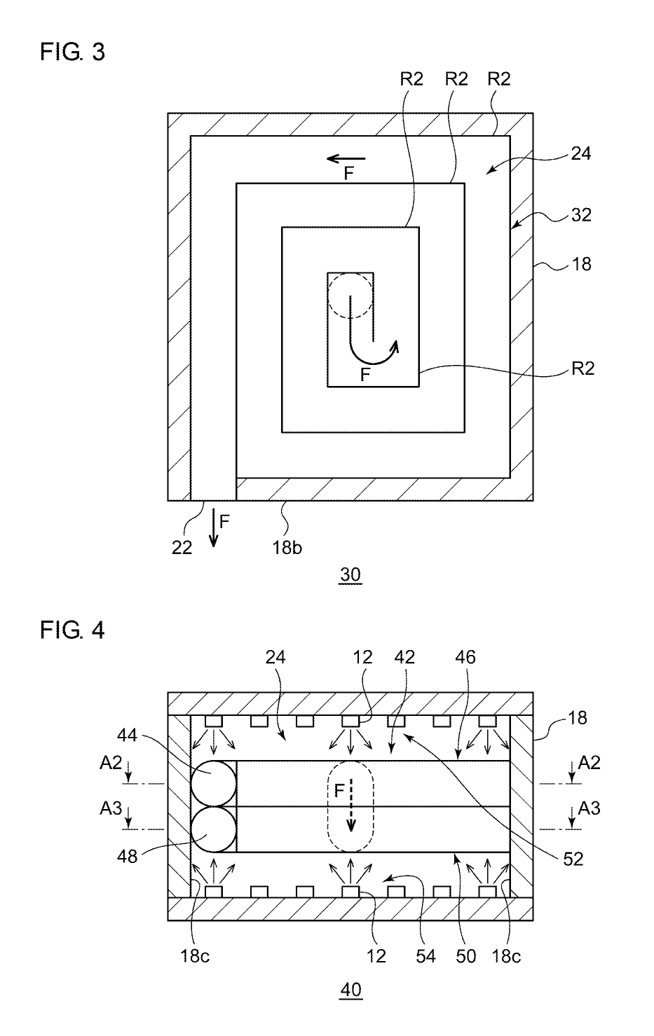

FIG. 3 is a horizontal cross-sectional view of a fluid sterilization apparatus 30 according to a second embodiment. In comparison with the fluid sterilization apparatus 10 according to the first embodiment, the main feature of the fluid sterilization apparatus 30 according to the second embodiment is that the structure of a flow passage 32 is different, and the other structures are substantially the same as those of the fluid sterilization apparatus 10. In the following embodiments, the same reference numerals will be given to the same structures as those of respective fluid sterilization apparatuses according to the embodiments, and the explanation thereof will be appropriately omitted.

The flow passage 32 in the fluid sterilization apparatus 30 is provided in the downward direction toward a sterilization chamber 24 inside a housing 18 from an inlet port 20 and has a shape where the flow passage 32 is wound along the outer circumference of the flow passage 32 while being bent repeatedly at a right angle in a winding passage R2 after changing the direction to the horizontal direction inside the sterilization chamber 24. The flow passage 32 formed in this manner is arranged with almost no gap in the sterilization chamber 24 whose horizontal cross-sectional surface is square, compared to the flow passage 16 of the fluid sterilization apparatus 10 shown in FIG. 2. This allows the length of the flow passage 32 in the sterilization chamber 24 to be longer.

### Third Embodiment

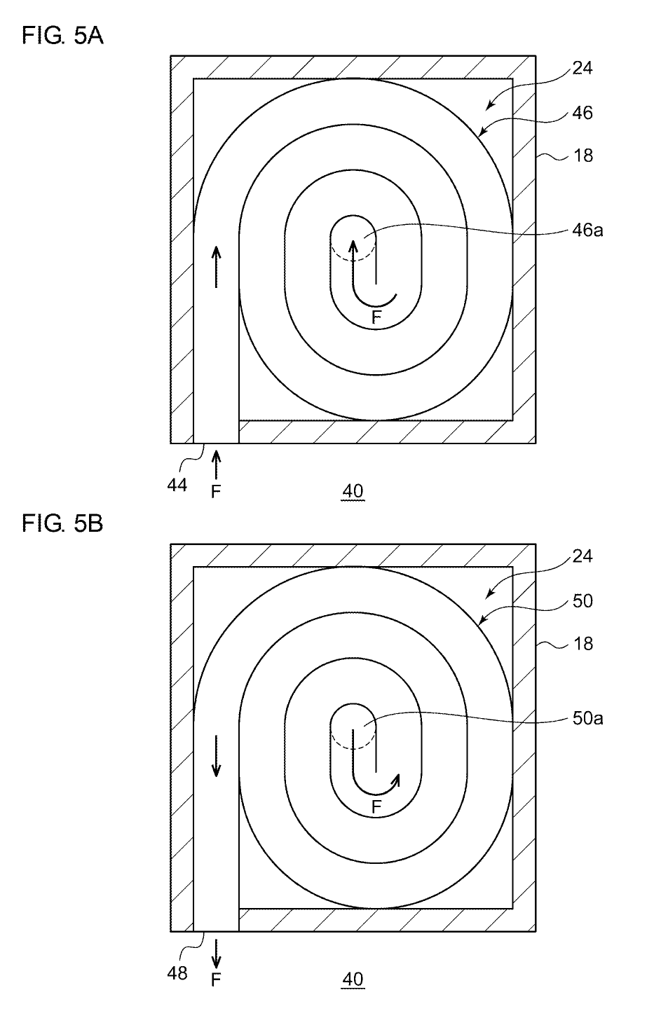

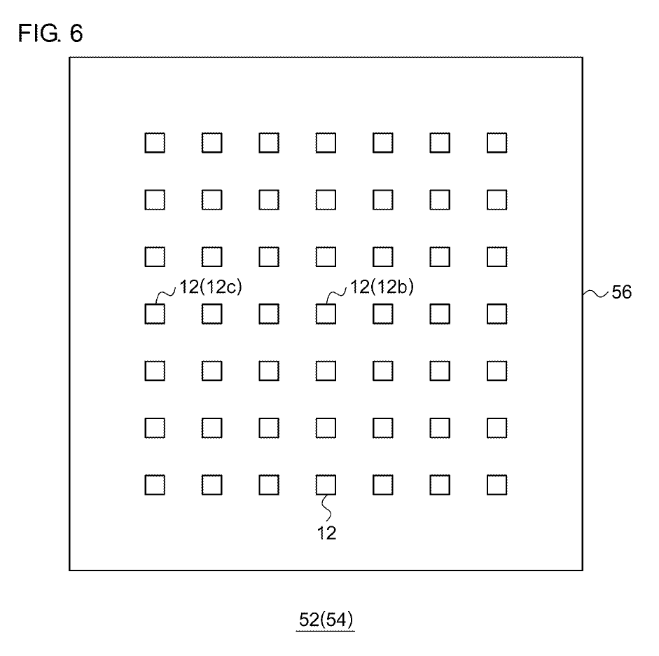

FIG. 4 is a schematic diagram showing a schematic configuration of a fluid sterilization apparatus according to a third embodiment. FIG. 5A is a cross-sectional view taken along A2-A2 of FIG. 4, and FIG. 5B is a cross-sectional view taken along A3-A3 of FIG. 4. FIG. 6 is a diagram showing the layout of a light source of the fluid sterilization apparatus according to the third embodiment.

A flow passage 42 in a fluid sterilization apparatus 40 has a first flow passage 46 on a first stage having an inlet port 44 for a fluid F and a second flow passage 50 on a second stage having an outlet port 48 for the fluid. An end portion 46a on the side opposite to the inlet port 44 of the first flow passage 46 and an end portion 50a on the side opposite to the outlet port 48 of the second flow passage 50 are connected to each other. This allows the fluid F passing through the second flow passage 50 or the first flow passage 46 to be irradiated with ultraviolet light that has passed through the first flow passage 46 or the second flow passage 50.

Further, the first flow passage 46 is arranged above the second flow passage 50 so as to overlap with the second flow passage 50. This allows the fluid F that has passed through the first flow passage 46 to flow into the second flow passage 50 due to the fluid's own weight.

The light source according to the present embodiment has a first light source 52 for irradiation with ultraviolet light from one side of the first flow passage 46 and a second light source 54 for irradiation with ultraviolet light from the other side of the second flow passage 50. This allows for irradiation with ultraviolet light from both sides of the flow passage 42. Thus, in comparison with irradiation with ultraviolet light from one side, the irradiation intensity of ultraviolet light for the fluid F passing through a predetermined interval can be higher, and the irradiation time can be shortened. In other words, since a flow passage that is arranged in a region irradiated with ultraviolet light can be shortened or the flow rate can be increased (the inner diameter of the flow passage can be decreased), the size of the apparatus can be reduced.

Further, on the inside of the housing 18, a reflective portion 18c, which reflects ultraviolet light emitted from the semiconductor light-emitting element 12, is provided. The reflective portion 18c is a reflective film or a reflective plate of a metal such as aluminum.

The flow passage on the first stage and the flow passage of the second stage (or a flow passage on a third or subsequent stage) do not need to have the same number of turns, and flow passages having different structures may be combined.

As shown in FIG. 6, in the first light source 52 and the second light source 54, a plurality of semiconductor light-emitting elements 12 are arranged in a seven-by-seven matrix on a circuit substrate 56. Thereby, even in a case of a semiconductor light-emitting element whose output is small by itself, arranging a plurality of semiconductor light-emitting elements allows sufficient sterilization performance to be achieved all through the flow passage 42. The plurality of semiconductor light-emitting elements 12 may be arranged in a line or a matrix of m by n (m is an integer of one or more and n is an integer of two or more).

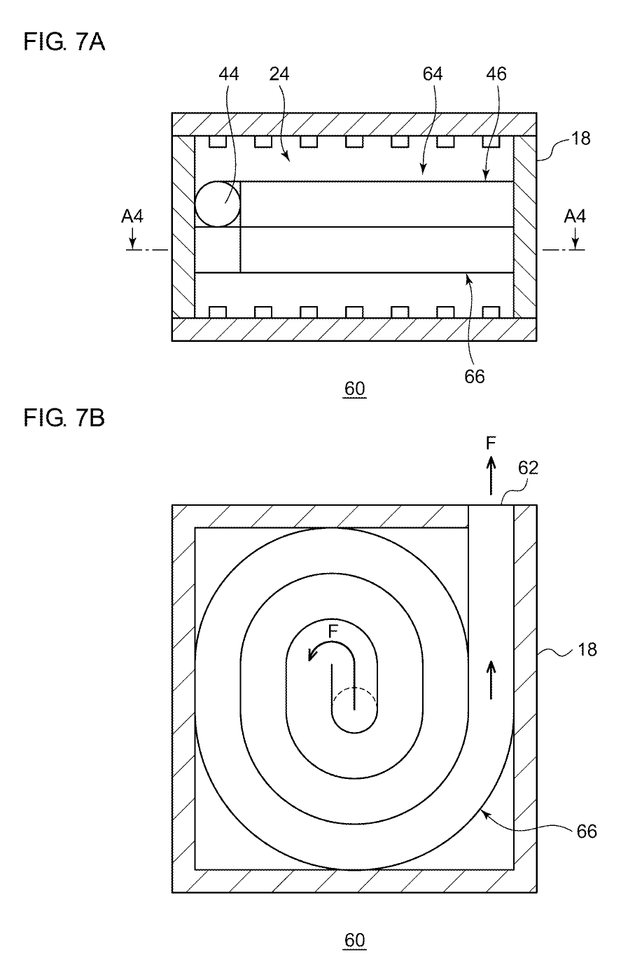

FIG. 7A is a schematic diagram showing a schematic configuration of a fluid sterilization apparatus according to an exemplary variation of the third embodiment, and FIG. 7B is a cross-sectional view taken along A4-A4 of FIG. 7A.

A fluid sterilization apparatus 60 shown in FIG. 7A is greatly different from the fluid sterilization apparatus 40 shown in FIG. 6 in that the position of an outlet port 62 is provided on the side opposite from an inlet port 44. A flow passage 64 has a first flow passage 46 on a first stage having the inlet port 44 for a fluid F and a second flow passage 66 on a second stage having the outlet port 62 for the fluid. This allows for the achievement of the fluid sterilization apparatus 60 where the inlet port 44 and the outlet port 62 are apart from each other. Thus, for example, interference between a housing unit that houses the fluid before the sterilization and a housing unit that houses the fluid after the sterilization become unlikely to occur.

### Fourth Embodiment

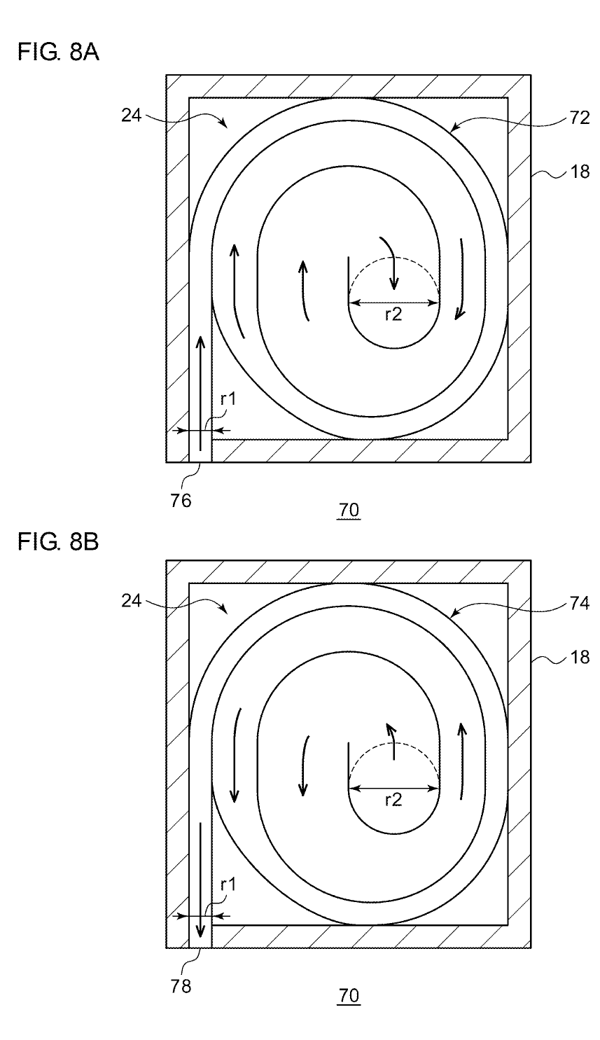

A fluid sterilization apparatus 70 according to a fourth embodiment is a fluid sterilization apparatus where the inner diameter of a flow passage is not constant. FIG. 8A is a top view of a first flow passage 72 in the fluid sterilization apparatus 70 according to the fourth embodiment, and FIG. 8B is a top view of a second flow passage 74 in the fluid sterilization apparatus 70 according to the fourth embodiment.

The first flow passage 72 is formed such that an inner diameter r2 in the middle of the first flow passage 72 (for example, the inner diameter at a central part of the sterilization chamber 24) is larger than an inner diameter r1 near an inlet port 76 for a fluid F. In the same way, the second flow passage 74 is formed such that an inner diameter r2 in the middle of the second flow passage 74 is larger than an inner diameter r1 of an outlet port for the fluid F. This allows the flow rate in the middle of a flow passage to be decreased. Thus, for example, by devising the arrangement of a light source so that the intensity of ultraviolet light irradiation in the middle of the flow passage is increased, the fluid can be efficiently sterilized. More specifically, in the fluid sterilization apparatus 70 according to the present embodiment, the flow rate of the fluid F can be decreased at the central part of the sterilization chamber 24, and the flow rate can be increased on the inner circumferential side of the sterilization chamber 24. This allows ultraviolet light irradiation time to be increased at the central part of the flow passage without lowering the processing speed. Changes in the inner diameter may include an increase and/or decrease in a linear manner and an increase and/or decrease in a stepwise manner and are not always limited to a monotonous increase and/or decrease.

Further, the first light source 52 (or the second light source 54) has a first semiconductor light-emitting element 12b arranged so as to face the central part of the first flow passage 72 (see FIG. 6) and a second semiconductor light-emitting element 12c arranged near the inlet port 76 (or near the outlet port 78) (see FIG. 6). The output of the second semiconductor light-emitting element 12c is larger than the output of the first semiconductor light-emitting element 12b. This allows the irradiation intensity at a place where the flow rate is high to be increased and the irradiation intensity at a place where the flow rate is low to be decreased. Thus, unevenness in the sterilization performance can be prevented all through the flow passage.

### Fifth Embodiment

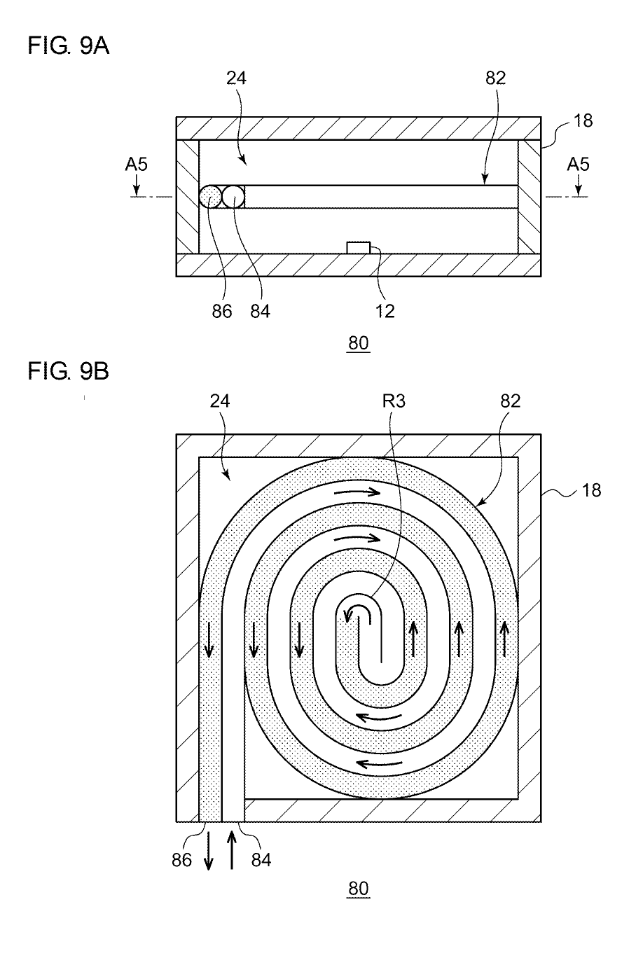

FIG. 9A is a schematic diagram showing a schematic configuration of a fluid sterilization apparatus according to a fifth embodiment, and FIG. 9B is a cross-sectional view taken along A5-A5 of FIG. 9A.

A flow passage 82 in a fluid sterilization apparatus 80 according to the fifth embodiment is arranged in a swirling shape and is formed of a continuous tube having a folded part R3 in the middle. The folded part R3 is arranged near the center of the fluid sterilization apparatus 80. Thereby, the single flow passage where both end portions of the flow passage 82 are located close to each other can be formed easily. In other words, an inlet port 84 and an outlet port 86 of the flow passage 82 can be arranged adjacent to each other. Further, even if flow passages in a plurality of stages as in the fluid sterilization apparatus 40 shown in FIG. 4 are not provided, a flow passage having almost the same length can be achieved, thus allowing for the thinning of the housing 18.

### Sixth Embodiment

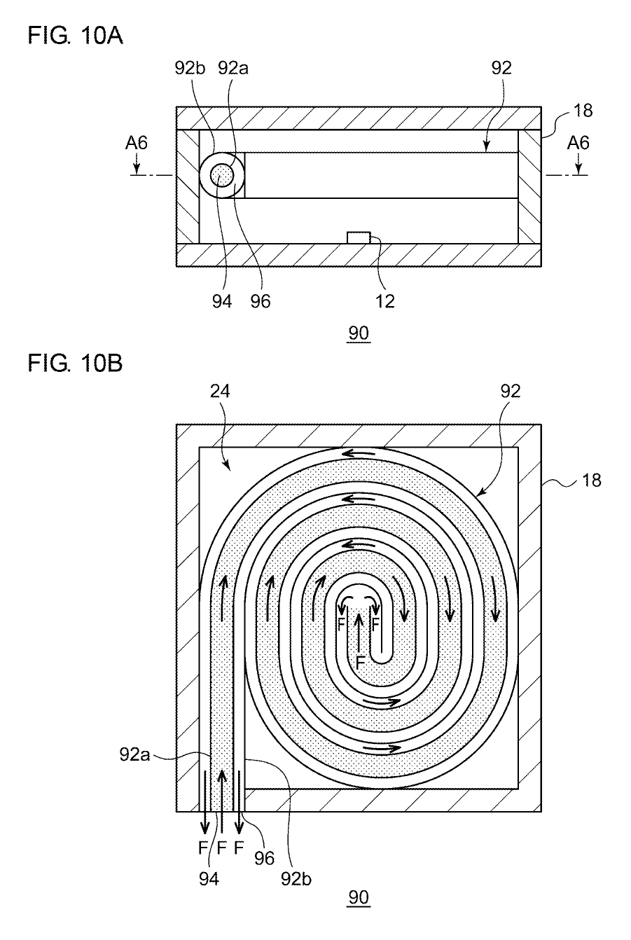

FIG. 10A is a schematic diagram showing a schematic configuration of a fluid sterilization apparatus according to a sixth embodiment, and FIG. 10B is a cross-sectional view taken along A6-A6 of FIG. 10A.

A flow passage 92 in a fluid sterilization apparatus 90 according to the sixth embodiment is arranged in a swirling shape and is formed of a tube having a double pipe structure formed of an inner pipe 92a and an outer pipe 92b. An end portion of the inner pipe 92a is open, and a fluid F that has flowed in from an inlet port 94, passed through the inner pipe 92a, and reached the central part of the sterilization chamber 24 flows between the outer pipe 92b and the inner pipe 92a toward an outlet port 96. Thereby, a single flow passage where both end portions of the flow passage 92 are integrated can be formed easily. In other words, the inlet port 94 and the outlet port 96 of the flow passage 92 can be arranged adjacent to each other. Further, even if flow passages in a plurality of stages as in the fluid sterilization apparatus 40 shown in FIG. 4 are not provided, a flow passage having almost the same length can be achieved, thus allowing for the thinning of the housing 18.

The present invention has been described by referring to each of the above-described embodiments. However, the present invention is not limited to the above-described embodiments only, and those resulting from any combination of them as appropriate or substitution are also within the scope of the present invention. Further, the combination of the embodiments or the process sequence thereof may be appropriately set or various modifications in design may be added to the embodiments based on the knowledge of the person skilled in the art. An embodiment having such modifications may be also included in the scope of the invention.

What is claimed is:

1. A fluid sterilization apparatus comprising:
a light source that has a semiconductor light-emitting element that emits ultraviolet light; and
a flow passage that is formed so as to allow a fluid subject to sterilization to pass through and allow the fluid to be irradiated with ultraviolet light,
wherein the flow passage is formed such that the flowing direction of the fluid changes in a plane that faces a light-emitting surface of the semiconductor light-emitting element.

2. The fluid sterilization apparatus according to claim 1,
wherein the flow passage has:
a first flow passage on a first stage having an inlet port for the fluid; and
a second flow passage on a second stage having an outlet port for the fluid,
wherein an end portion on the side opposite to the inlet port of the first flow passage and an end portion on the side opposite to the outlet port of the second flow passage are connected to each other.

3. The fluid sterilization apparatus according to claim 2, wherein the first flow passage is arranged above the second flow passage so as to overlap with the second flow passage.

4. The fluid sterilization apparatus according to claim 1,
wherein the light source has:
a first light source for irradiation with ultraviolet light from one side of the flow passage; and
a second light source for irradiation with ultraviolet light from the other side of the flow passage.

5. The fluid sterilization apparatus according to claim 1, further comprising a housing that houses the light source and the flow passage,
wherein, on the inside of the housing, a reflective portion that reflects ultraviolet light emitted from the semiconductor light-emitting element is provided.

6. The fluid sterilization apparatus according to claim 1, wherein the flow passage is formed such that at least a portion thereof has a spiral shape or a swirling shape.

7. The fluid sterilization apparatus according to claim 1, wherein the flow passage is formed of a tube.

8. The fluid sterilization apparatus according to claim 7, wherein the tube is made of a fluorine-based resin or a silicone based resin.

9. The fluid sterilization apparatus according to claim 1, wherein the flow passage is formed of a continuous tube having a folded part in the middle, the folded part being arranged near the center of the fluid sterilization apparatus.

10. The fluid sterilization apparatus according to claim 1, wherein the flow passage is formed such that the inner diameter of the flow passage in the middle of the flow passage is larger than the inner diameter of the flow passage near an inlet port or an outlet port for the fluid.
